(19) United States
(12) Patent Application Publication
(10) Pub. No.: US 2016/0110871 A1
(43) Pub. Date: Apr. 21, 2016

(54) APPARATUS AND METHOD FOR SUPPORTING IMAGE DIAGNOSIS

(71) Applicant: Samsung Electronics Co., Ltd., Suwon-si (KR)

(72) Inventors: Hyo A. KANG, Seoul (KR); Won Sik KIM, Gunpo-si (KR)

(73) Assignee: SAMSUNG ELECTRONICS CO., LTD., Suwon-si (KR)

(21) Appl. No.: 14/887,901

(22) Filed: Oct. 20, 2015

(30) Foreign Application Priority Data
Oct. 21, 2014 (KR) 10-2014-0142896

(51) Int. Cl. G06T 7/00; G06K 9/52; A61B 8/08; G06T 7/20; A61B 8/00; G06K 9/62; G06T 7/60

(52) U.S. Cl. CPC G06T 7/0044; G06T 7/0014; G06K 9/6215; G06K 9/52; G06T 7/60; G06T 7/204; A61B 8/4254; A61B 8/469; A61B 8/466; A61B 8/08; G06K 2009/4666

(57) ABSTRACT

An apparatus and method for supporting an image diagnosis. The apparatus may include a location determiner configured to determine a relative location of a probe with respect to a subject's body based on absolute location information and angle information of the probe, a mapper configured to map, to a three-dimensional model, a current image acquired through the probe using the relative location of the probe, and a presenter configured to output a three-dimensional model where the mapped current image and a pre-captured part are marked.

### CROSS-REFERENCE TO RELATED APPLICATION(S)

This application claims the benefit under 35 U.S.C. §119(a) of Korean Patent Application No. 10-2014-0142896, filed on Oct. 21, 2014, in the Korean Intellectual Property Office, the entire disclosure of which is incorporated herein by reference for all purposes.

### BACKGROUND

1. Field

The following description relates to an image diagnosis technology, and to an apparatus and method for supporting an image diagnosis.

2. Description of Related Art

A currently-used probe of an ultrasound device has some limitations in acquiring an ultrasound image. For example, after acquiring the image, information on a location where the image is captured and a direction of the probe at a point in time when the image is captured is not known.

Thus, anatomically checking an image of a desired position when the image is reviewed, or capturing the same area when the desired position is re-examined, depend on a doctor's experience.

### SUMMARY

In one general aspect, there is provided an apparatus for supporting an image diagnosis including a location determiner configured to determine a relative location of a probe with respect to a subject's body based on absolute location information and angle information of the probe, a mapper configured to map, to a three-dimensional model, a current image acquired through the probe using the relative location of the probe, and a presenter configured to output a three-dimensional model where the mapped current image and a pre-captured part are marked.

The location determiner may be further configured to acquire the absolute location information with respect to a specific area of the subject's body, to compare the acquired absolute location information and the absolute location information of the probe, and to determine the relative location of the probe based on a result of the comparison.

The three-dimensional model may include the subject's organ or tissue.

The mapper may be configured to map the acquired image to the three-dimensional model based on a degree of similarity between the current image and a previous stored image mapped to the three-dimensional model.

The apparatus may include an absolute location and angle acquirer configured to acquire the absolute location information and the angle information of the probe and comprising at least a sensor built in the probe.

The apparatus may include a guide information generator configured to generate guide information for searching a specific area based on a result of the mapping and location and capturing angle information of a pre-stored specific area.

The guide information may include at least one of a movement direction, a moving distance, or a capturing angle of the probe.

The presenter may output the guide information to a screen.

The specific area may include at least one of a non-captured area, an area that a user selects, or a preset region of interest (ROI).

The apparatus may include a storage configured to store, in response to the specific area being captured, at least one of a captured image of the specific area, or a relative location and an angle of the probe at a time the image is captured.

In another general aspect, there is provided a method of supporting an image diagnosis including a processor performing operations of determining a relative location of a probe with respect to a subject's body based on absolute location information and angle information of the probe, mapping, to a three-dimensional model, a current image acquired through the probe using the relative location of the probe, and outputting a three-dimensional model where the mapped current image and a pre-captured part are marked.

The determining of the relative location of the probe may include acquiring the absolute location information with respect to a specific area of the subject's body, comparing the acquired absolute location information and the absolute location information of the probe, and determining the relative location of the probe based on a result of the comparison.

The three-dimensional model may include the subject's organ or tissue.

The mapping of the current image may include mapping the acquired image to the three-dimensional model based on a degree of similarity between the current image and a previous stored image mapped to the three-dimensional model.

The method may include acquiring the absolute location information and the angle information of the probe through a sensor built in the probe.

The method may include guide information for searching a specific area based on a result of the mapping and location and capturing angle information of a pre-stored specific area.

The guide information may include at least one of a movement direction, a moving distance, or a capturing angle of the probe.

The method may include outputting the guide information to a screen.

The specific area may include at least one of a non-captured area, an area that a user selects, or a preset region of interest (ROI).

The method may include storing, in response to the specific area being captured, at least one of a captured image of the specific area, or a relative location and an angle of the probe of a point in time the image is captured.

The three-dimensional model may include a personalized organ or tissue model generated in advance based on the subject's information.

The determining of the relative location may include determining of the relative location based on comparing the personalized three-dimensional model and the current image.

The guide information may include at least one of vibration or sound.

In another general aspect, there is provided a method of supporting an image diagnosis, including a processor performing operations of determining a relative location of a probe based on an absolute location and an angle of the probe, mapping a current image acquired through the probe to a three-dimensional model based on the relative location of the probe and similarity between the current image and a pre-stored image, generating guide information for searching a specific area based on the mapping and information of a specific area, and outputting the guide information and a three-dimensional model with the mapped current image and a pre-captured part.

Other features and aspects may be apparent from the following detailed description, the drawings, and the claims.

### BRIEF DESCRIPTION OF THE DRAWINGS

FIG. 1 is a diagram illustrating an example of an apparatus for supporting an image diagnosis.

FIG. 2 is a diagram illustrating an example of an apparatus 12 for supporting an image diagnosis of FIG. 1.

FIG. 3 is a diagram illustrating an example of an apparatus 12 for supporting an image diagnosis of FIG. 1.

FIG. 4 is a diagram illustrating an example of a screen that is output by a screen output.

FIG. 5 is a diagram illustrating another example of a screen that is output by a screen output.

FIG. 6 is a diagram illustrating a method of supporting an image diagnosis.

FIG. 7 is a diagram illustrating a method of supporting an image diagnosis.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses, and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

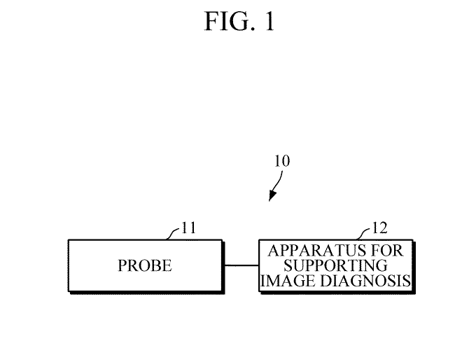

FIG. 1 is a diagram illustrating an example of an apparatus for supporting an image diagnosis. Referring to FIG. 1, a apparatus 10 for supporting an image diagnosis may include a probe 11 and an apparatus 12 for supporting an image diagnosis. The probe 11 may acquire image data by irradiating an ultrasound to an object and receiving an echo ultrasound reflected from the object.

The probe 11 may sense its absolute location and angle by using a sensor built in the inside or the outside. The sensor may include various sensors that measure a location and/or an angle, such as, for example, a geomagnetic sensor, a GPS sensor, an acceleration sensor, and an angle sensor. The sensor may be built inside the probe 11, and may be configured as an attachable separate module to be attached to the outside of the probe.

An apparatus 12 for supporting an image diagnosis may receive the image data from the probe 11, form an image, and output the formed image to a screen (not shown).

The apparatus 12 may determine the relative location of the probe with respect to a patient's body on a basis of information of the absolute location and angle of the probe 11 when the image is captured. The apparatus 12 may map a current image acquired through the probe to a three-dimensional body model based on the determination result.

The three-dimensional body model is a model where an entire or part of a human body is displayed in three dimensions. For example, the three-dimensional body model may be a model showing a human's organ or tissue. The three-dimensional body model may be a standard organ model, a standard tissue model, or a personalized organ model or tissue model, which is generated in advance based on information of a patient.

The apparatus 12 may output, to a screen based on the mapping result, a three-dimensional body model where the currently captured part and the pre-captured part are marked.

The apparatus 12 may generate and output guide information for searching a specific area using the mapping result and information on a pre-stored specific area, such as, for example, the relative location of the specific area, and the angle of the probe of a point in time the specific area is captured. The specific area may include areas such as, for example, an uncaptured area, a pre-set region of interest (ROI), and an area that a user selects. In addition, the guide information may include information such as, for example, a movement direction, a moving distance, and an angle of the probe.

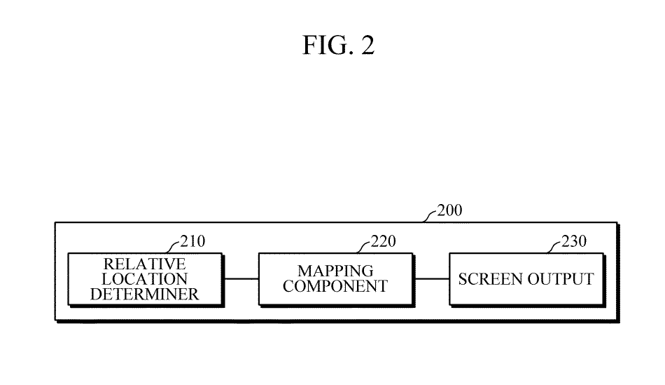

FIG. 2 is a diagram illustrating an example of an apparatus 12 for supporting an image diagnosis of FIG. 1. Referring to FIG. 2, an apparatus 200 for supporting an image diagnosis may include a relative location determiner 210, a mapping component (also referred to as a mapper) 220, and a screen output (also referred to as a presenter) 230.

The relative location determiner 210 may determine a relative location of the probe 11 with respect to a patient's body. In an example, the relative location determiner 210 may determine the relative location of the probe 11 with respect to a patient's body based on absolute location information and angle information of the probe 11.

For example, the relative location determiner 210 may acquire the absolute location information with respect to a specific area of the patient's body, which is set as a standard point. The relative location determiner 210 may compare the acquired absolute location information with respect to the specific area of a patient's body and the absolute location information of the probe. The relative location determiner 210 may determine the relative location of the probe 11 with respect to the patient's body based on the result of the comparison and angle information of the probe 11.

The standard point may indicate a point that is a standard for determining the relative location with respect to the area of a patient's body. The absolute location information with respect to the specific area of a patient's body that has been set as the standard point may be acquired by positioning, by a user, the probe 11 that is built with a positioning sensor on the area of the body.

In another example, the relative location determiner 210 may determine the relative location of the probe 11 with respect to the patient's body by comparing the pre-stored image and the current image that is acquired through the probe 11.

For example, the relative location determiner 210 may determine a degree of similarity between the pre-stored image and the current image, and when a previous image exists with the degree of similarity greater than a threshold, the relative location determiner 210 may determine the relative location of the probe 11 of a point in time the previous image is captured, as the relative location of the probe 11 of a point in time the current image is captured.

In yet another example, the relative location determiner 210 may determine the relative location of the probe 11 with respect to the patient's body by comparing the standard image and the current image that has been acquired through the probe 11. Here, the standard image is an image that has been acquired after capturing a specific area of the patient's body with a precise characteristic that is a standard for determining the relative location of the probe 11, and may be captured before or after the examination. The area of a patient's body with the precise characteristic may indicate areas that are expected to be positioned at a relatively fixed location on the body, such as a navel, a face, and the like.

For example, the relative location determiner 210 may compare the standard image and the current image that has been acquired through the probe 11, identify the location of the specific area of the body within the current image, and determine the relative location of the probe 11 based on the identified location.

The absolute location information and the angle information of the probe may be acquired by a sensor built in the probe 11. The sensor may include various sensors that measure a location and/or an angle, such as, for example, a geomagnetic sensor, a GPS sensor, an acceleration sensor, and an angle sensor.

The mapping component 220 may map the image acquired through the probe 11 to a three-dimensional body model.

The three-dimensional body model is a model where an entire or part of a body is displayed in three dimensions. In an example, the three-dimensional model may show a human's organ or tissue. In another example, the three-dimensional body model may be a standard organ model, a standard tissue model, or a personalized organ model or tissue model, which is generated in advance based on information on a patient.

In an example, the mapping component 220 may map a current image to a three-dimensional body model based on the relative location of the probe 11, which has been determined by the relative location determiner 210.

In another example, the mapping component 220 may map the current image to the three-dimensional body model based on a degree of similarity between the previous image, which is stored as being mapped to the three-dimensional body model, and the current image. For example, the mapping component 220 may determine the previous image stored as being mapped to the three-dimensional body model and the image acquired through the probe 11. When a previous image is present with the degree of similarity greater than a preset threshold, the mapping component 220 may map the current image to the three-dimensional body model location where the previous image is mapped.

In another example, the mapping component 11 may map the current image to the three-dimensional body model in consideration of both the relative location of the probe 11 and the determination result of the degree of similarity between the current image acquired through the probe 11 and the pre-stored image.

The screen output 230 may output the current image to a screen. The screen output 230 may output the three-dimensional body model, to the screen based on the mapping result of the mapping component 220. The three-dimensional body model output on the screen may mark a currently captured part and a pre-captured part. Accordingly, a user may know the location of the captured part, the pre-captured part, and a non-captured part of the current image.

The screen output 230 may output information on the relative location of the probe 11 at the moment when the current image is captured.

In an example, the screen output 230 may output a three-dimensional body model where the currently captured part and the pre-captured part are marked, and the information on the relative location of the probe at a point in time the current image is captured. The three-dimensional body model may be output to a screen area excluding the area where the current image has been output. In another example, the three-dimensional body model may be output to another screen different from the screen where the current image has been output so that a user is not interrupted during a diagnosis. Also, the screen output 230 may emphasize the captured part of the current image, which is displayed on the three-dimensional body model, through techniques, such as, for example, highlighting, color and blinking.

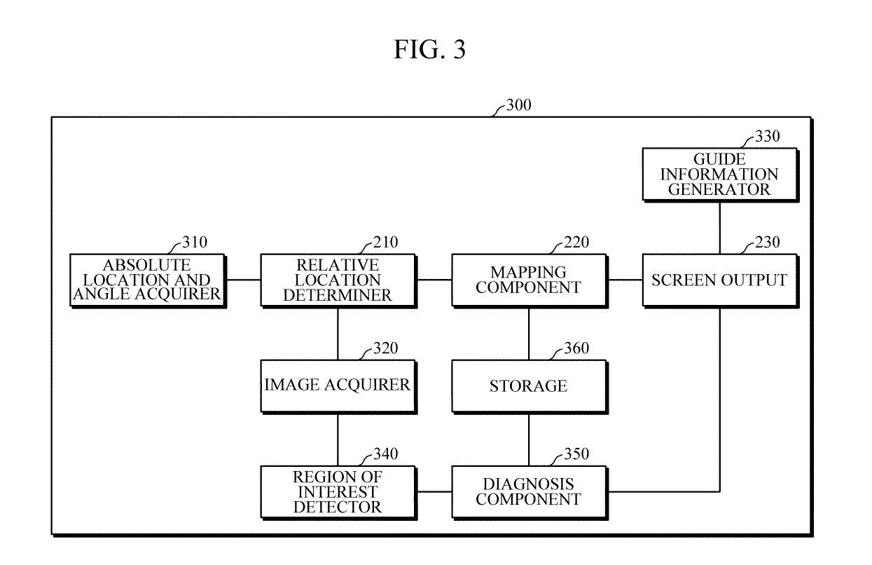

FIG. 3 is a diagram illustrating an apparatus 12 for supporting an image diagnosis of FIG. 1. Some of the components shown in FIG. 3 have been described with reference to FIGS. 1-2. The above description of FIGS. 1-2, is also applicable to FIG. 3, and is incorporated herein by reference. Thus, the above description may not be repeated here.

Referring to FIG. 3, in addition to the components of an apparatus 200 for supporting an image diagnosis of FIGS. 2 (210, 220, and 230), an apparatus 300 for supporting an image diagnosis according to another example may selectively include an absolute location and angle acquirer 310, an image acquirer 320, a guide information generator 330, an Region of Interest (ROI) detector 340, a diagnosis component 350, and a storage 360.

As described above, the absolute location and angle acquirer 310 may acquire an absolute location and angle of a probe 11 through a sensor built in the probe 11.

The image acquirer 320 may acquire a medical image of a patient through the probe 11. Here, the medical image may be an ultrasound image acquired in real time by a frame unit through the probe 11. For example, the image acquirer 320 may form the medical image by using image data received from the probe 11. Also, the image acquirer 320 may delete and correct basic noise with respect to the acquired image.

To search a specific area of a patient's body, the guide information generator 330 may generate guide information such as, for example, a movement direction, a moving distance, and an angle of the probe 11 based on a mapping result of a mapping component 220, location information of the pre-stored specific area, and capturing angle information. Here, the specific area may include areas such as, for example a non-captured area, a pre-set ROI, and an area that a user selects. The generated guide information may be provided to a user in a form of images, vibrations, and sounds.

If the guide information is provided to a user in a form of an image, the guide information may be output to the screen through the screen output 230. Here, the screen output 230 may output the guide information to a screen area excluding the area where the current image has been output or to another screen different from the screen where the current image has been output so that a user is not interrupted during a diagnosis.

The ROI detector 340 may analyze the image acquired in real time by the image acquirer 320 and detect its ROI. For example, the ROI detector 340 may detect the ROI by using an automatic lesion detection algorithm, such as, for example, AdaBoost, DPM (Deformable Part Models), DNN (Deep Neural Network), CNN (Convolutional Neural Network), and Sparse coding.

The diagnosis component 350 may diagnose the ROI detected from the ROI detector 340. For example, the diagnosis component 350 may diagnose the detected ROI using a lesion classification algorithm. The lesion classification algorithm of classifying a lesion may include algorithms such as, for example, SVM (Support Vector Machine), Decision Tree, DBN (Deep Belief Network), and CNN (Convolutional Neural Network).

The result of detecting the ROI and the diagnosis may be output through the screen output 230.

The storage 360 may store diverse medical information on a patient. For example, the storage 360 may store information, such as, for example, the pre-captured image, absolute location information, relative location information, angle information of the probe of a point in time the image is captured, detection result information and diagnosis information of the ROI, a relative location and capturing angle information of the preset ROI, a three-dimensional body model where the current image is mapped, and guide information.

If a specific area, such as, for example, a non-captured area, a pre-set ROI, and an area that a user selects is captured, the storage 360 may automatically store an image of the captured specific area, and relative/absolute location information of the probe of a point in time the specific area is captured and its angle information.

The storage 360 may include flash memory type, hard disk type, multimedia card micro type, card-typed memory (e.g., Secured Digital (SD) or Extreme Digital (XD) memory), Random Access Memory (RAM), Static Random Access Memory (SRAM), Read Only Memory (ROM), Electrically Erasable Programmable Read Only Memory (EEPROM), Programmable Read Only Memory (PROM), Magnetic Memory (MRAM), CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, magnetic disk, floppy disks, magneto-optical data storage devices, optical data storage devices, hard disks, solid-state disks, and any device known to one of ordinary skill in the art that is capable of storing any image and associated data, data files, and data structures in a non-transitory manner and providing the instructions or software.

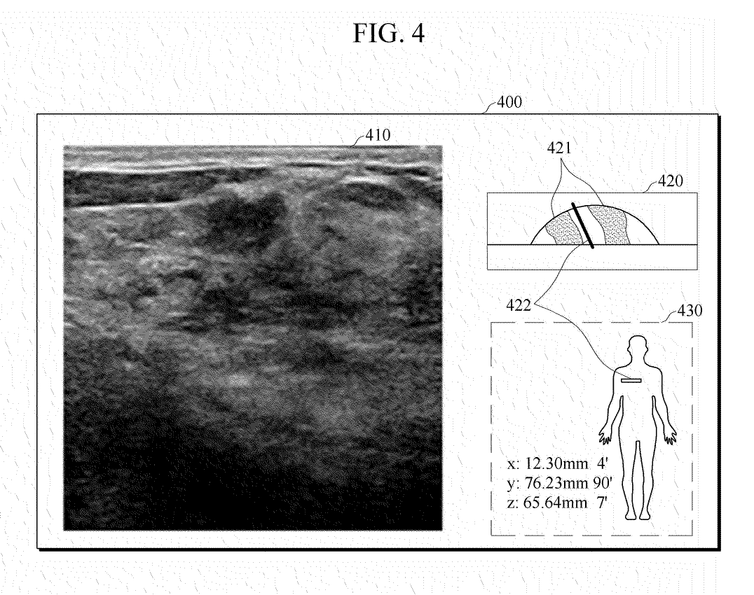

FIG. 4 is a diagram illustrating an example of a screen that is output by a screen output. Referring to FIG. 4, a screen output 230 may output a current image 410, a three-dimensional body model 420, and relative location information 430 of a probe at the moment when the current image 410 is captured.

As illustrated in FIG. 4, a currently captured part 422 and a pre-captured part 421 are marked in the three-dimensional body model 420. The pre-captured part 421 may be marked in a different color, transparency, and a type of line (e.g., a dotted line and a solid line, etc.) to be separable from a non-captured part.

The three-dimensional body model 420 may have a view angle and a view direction to be changed according to a user's command, so that the user can observe from a desired view, and a specific area of the three-dimensional body model 420 may be expanded or reduced.

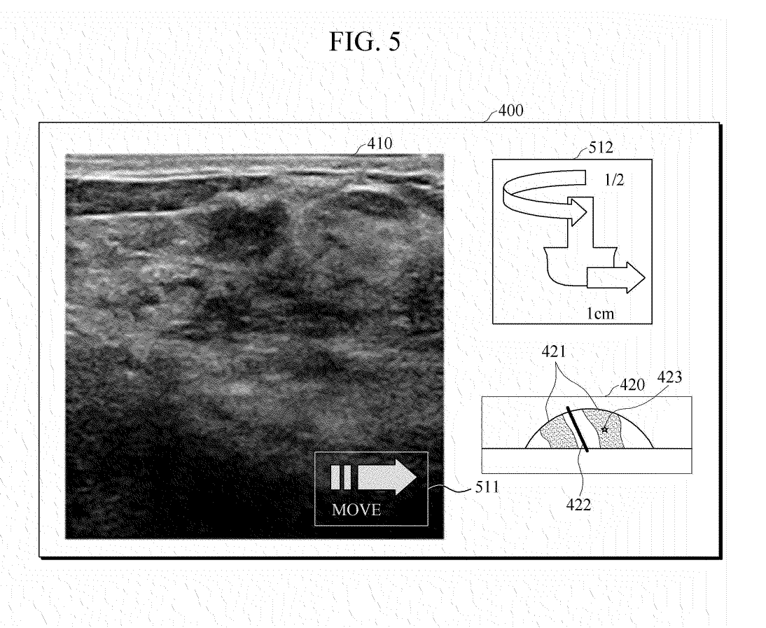

FIG. 5 is a diagram illustrating another example of a screen that is output by a screen output.

Referring to FIG. 5, a screen output 230 outputs a current image 410, a three-dimensional body model 420, and guide information 511 and 512 to a screen 400.

As illustrated in FIG. 5, a captured part 422 of the current image 410, a pre-captured part 421, and a pre-set ROI 423 are marked in the three-dimensional body model 420.

In the illustrated example, the ROI 423 is marked as a star shape in the three-dimensional body model 420. In other non-limiting examples, the screen output 230 may show a location of the ROI 423 by displaying the ROI 423 with a bounding box or displaying a dot or a cross line, etc., in the center of the ROI 423.

Here, the guide information 511 and 512 is information for searching the ROI 423, and may include a movement direction, a moving distance, and a capturing angle of a probe.

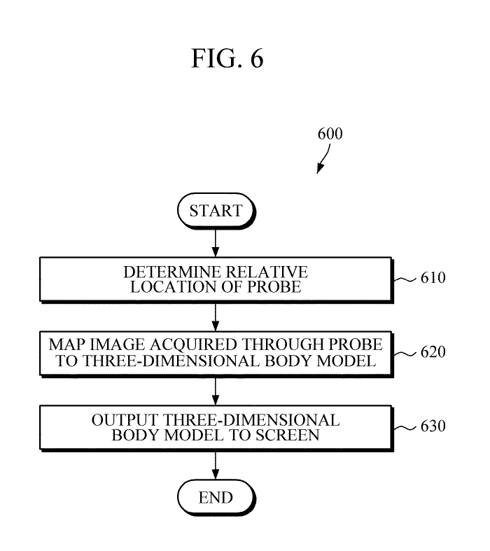

FIG. 6 is a diagram illustrating a method of supporting an image diagnosis. The operations in FIG. 6 may be performed in the sequence and manner as shown, although the order of some operations may be changed or some of the operations omitted without departing from the spirit and scope of the illustrative examples described. Many of the operations shown in FIG. 6 may be performed in parallel or concurrently. The above description of FIGS. 1-5, is also applicable to FIG. 6, and is incorporated herein by reference. Thus, the above description may not be repeated here.

Referring to FIG. 6, a method 600 of supporting an image diagnosis first determines relative location of a probe with respect to a patient's body in 610.

For example, an apparatus 200 for supporting an image diagnosis may determine a relative location of a probe 11 with respect to a patient's body based on relative location information and angle information. In another example, the apparatus 200 may determine the relative location of the probe 11 with respect to the patient's body by comparing a pre-stored image and a current image that is acquired through the probe 11.

In 620, the image acquired through the probe 11 is mapped to a three-dimensional body model. The three-dimensional body model is model where an entire or part of a body is displayed in three dimensions, and may be a three-dimensional model showing a human's organ or tissue. The three-dimensional body model may be a standard organ model, a standard tissue model, or a personalized organ model or tissue model, which is generated in advance based on information on a patient.

For example, the apparatus 200 may map the current image to a three-dimensional body model based on the relative location of the probe, which has been determined by a relative location determiner 210. In another example, the apparatus 200 may map the current image to the three-dimensional body model based on a degree of similarity between the previous image, which is stored as being mapped to the three-dimensional body model, and the current image. In another example, the apparatus 200 may map the current image to the three-dimensional body model in consideration of both the relative location of the probe 11 and the determination result of the degree of similarity between the image acquired through the probe 11 and the pre-stored image.

In 630, the apparatus 200 outputs, to a screen based on the mapping result, the three-dimensional body model, where the currently captured part and the pre-captured part are marked in 630. Here, the apparatus 200 may output information with respect to the relative location of the probe 11 at the moment when the current image is captured.

For example, the apparatus 200 may output a three-dimensional body model where the currently captured part and the pre-captured part are marked, and the information on the relative location of the probe of a point in time the current image is captured, to a screen area excluding the area where the current image has been output or to another screen different from the screen where the current image has been output so that a user is not interrupted during a diagnosis.

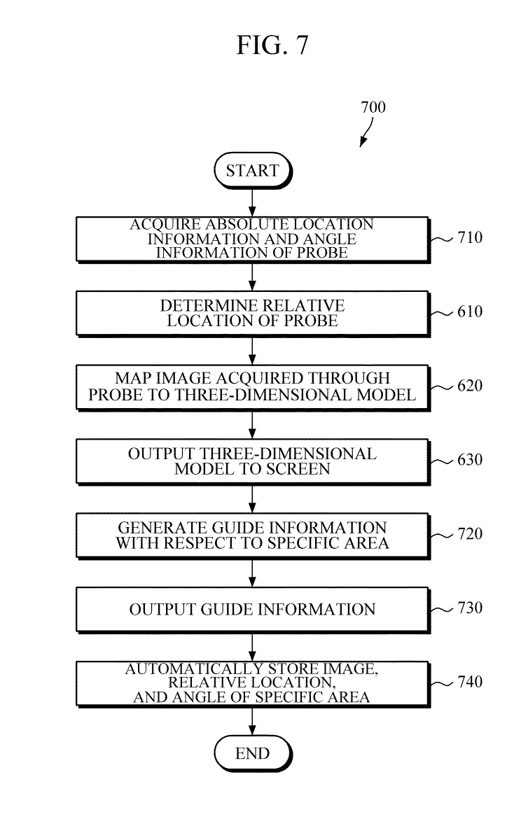

FIG. 7 is a diagram illustrating a method of supporting an image diagnosis. The operations in FIG. 7 may be performed in the sequence and manner as shown, although the order of some operations may be changed or some of the operations omitted without departing from the spirit and scope of the illustrative examples described. Many of the operations shown in FIG. 7 may be performed in parallel or concurrently. The above description of FIGS. 1-6, is also applicable to FIG. 7, and is incorporated herein by reference. Thus, the above description may not be repeated here.

Referring to FIG. 7, in addition to a method 600 of supporting imaging an image diagnosis of FIG. 6, a method 700 of supporting an image diagnosis may selectively further include acquiring relative location information and angle information of the probe in 710, generating guide information in 720, outputting guide information in 730, and automatically storing information in 740.

In 710, a relative location and an angle of the probe 11 are acquired through a sensor built in the probe 11.

In generating the guide information in 720, the guide information for searching a specific area of a patient's body is generated. Here, the specific area may include areas such as, for example, a non-captured area, a pre-set ROI, and an area that a user selects

For example, to search the specific area of the patient's body, an apparatus 300 for supporting an image diagnosis may generate guide information including information such as, for example, a movement direction, a moving distance, and an angle of the probe 11 based on the mapping result of the mapping component 220, location information of the pre-stored specific area, and capturing angle information. The apparatus 300 outputs the generated guide information to a screen.

For example, the apparatus 300 may output the guide information to a screen area excluding the area where the current image has been output or to another screen different from the screen where the current image has been output so that a user is not interrupted during a diagnosis.

In other examples, the guide information may be provided to a user in a form of sounds or vibrations as well as images.

In automatically storing in 740, if the specific area (e.g., a non-captured area, a pre-set ROI, and an area that a user selects) is captured, the apparatus 300 may store information such as, for example, an image of the captured specific area, and relative/absolute location information and angle information of the probe of a point in time the specific area is captured.

The apparatuses, units, modules, devices, and other components illustrated that perform the operations described herein are implemented by hardware components. Examples of hardware components include controllers, sensors, generators, drivers and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array (FPGA), a programmable logic array, a microprocessor, an application-specific integrated circuit (ASIC), or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 7-7 that perform the operations described herein are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. An apparatus for supporting an image diagnosis, comprising:
a location determiner configured to determine a relative location of a probe with respect to a subject's body based on absolute location information and angle information of the probe;
a mapper configured to map, to a three-dimensional model, a current image acquired through the probe using the relative location of the probe; and
a presenter configured to output a three-dimensional model where the mapped current image and a pre-captured part are marked.

2. The apparatus of claim 1, wherein the location determiner is further configured to acquire the absolute location information with respect to a specific area of the subject's body, to compare the acquired absolute location information and the absolute location information of the probe, and to determine the relative location of the probe based on a result of the comparison.

3. The apparatus of claim 1, wherein the three-dimensional model comprises the subject's organ or tissue.

4. The apparatus of claim 1, wherein the mapper is further configured to map the acquired image to the three-dimensional model based on a degree of similarity between the current image and a previous stored image mapped to the three-dimensional model.

5. The apparatus of claim 1, further comprising an absolute location and angle acquirer configured to acquire the absolute location information and the angle information of the probe and comprising at least a sensor built in the probe.

6. The apparatus of claim 1, further comprising a guide information generator configured to generate guide information for searching a specific area based on a result of the mapping and location and capturing angle information of a pre-stored specific area.

7. The apparatus of claim 6, wherein the guide information comprises at least one of a movement direction, a moving distance, or a capturing angle of the probe.

8. The apparatus of claim 6, wherein the presenter is further configured to output the guide information to a screen.

9. The apparatus of claim 6, wherein the specific area comprises at least one of a non-captured area, an area that a user selects, or a preset region of interest (ROI).

10. The apparatus of claim 6, further comprising:
a storage configured to store, in response to the specific area being captured, at least one of:
a captured image of the specific area; or
a relative location and an angle of the probe at a time the image is captured.

11. A method of supporting an image diagnosis, comprising:
a processor performing operations of:
determining a relative location of a probe with respect to a subject's body based on absolute location information and angle information of the probe;
mapping, to a three-dimensional model, a current image acquired through the probe using the relative location of the probe; and
outputting a three-dimensional model where the mapped current image and a pre-captured part are marked.

12. The method of claim 11, wherein the determining of the relative location of the probe comprises:
acquiring the absolute location information with respect to a specific area of the subject's body;
comparing the acquired absolute location information and the absolute location information of the probe; and
determining the relative location of the probe based on a result of the comparison.

13. The method of claim 11, wherein the three-dimensional model comprises the subject's organ or tissue.

14. The method of claim 11, wherein the mapping of the current image comprises mapping the acquired image to the three-dimensional model based on a degree of similarity between the current image and a previous stored image mapped to the three-dimensional model.

15. The method of claim 11, further comprising:
acquiring the absolute location information and the angle information of the probe through a sensor built in the probe.

16. The method of claim 11, further comprising:
generating guide information for searching a specific area based on a result of the mapping and location and capturing angle information of a pre-stored specific area.

17. The method of claim 16, wherein the guide information comprises at least one of a movement direction, a moving distance, or a capturing angle of the probe.

18. The method of claim 16, further comprising:
outputting the guide information to a screen.

19. The method of claim 16, wherein the specific area comprises at least one of a non-captured area, an area that a user selects, or a preset region of interest (ROI).

20. The method of claim 16, further comprising:
storing, in response to the specific area being captured, at least one of:
a captured image of the specific area; or
a relative location and an angle of the probe of a point in time the image is captured.
